(19) United States
(12) Patent Application Publication
(10) Pub. No.: US 2023/0324295 A1
(43) Pub. Date: Oct. 12, 2023

(54) ANALYZER AND METHOD FOR ANALYZING A SAMPLE

(71) Applicant: i-Abra Limited, Toddington Bedfordshire (GB)

(72) Inventors: Greg Compton, Toddington Bedfordshire (GB); Peter Saunderson, Toddington Bedfordshire (GB)

(21) Appl. No.: 18/024,518

(22) PCT Filed: Sep. 3, 2021
(86) PCT No.: PCT/GB2021/052274

§ 371 Date: Mar. 3, 2023

(30) Foreign Application Priority Data
Sep. 4, 2020 (GB) 2013961.4
Sep. 9, 2020 (GB) 2014178.4

(51) Int. Cl. G01N 21/41; B01L 3/00; B01L 9/00

(52) U.S. Cl. CPC G01N 21/41; B01L 3/5029; B01L 9/52; B01L 2300/042; B01L 2300/0822

(57) ABSTRACT

An analyzer and method of analyzing a sample are provided. The method includes providing a sample on a substrate, and irradiating the sample with at least three incident beams of light. Each of the incident beams contains light of a different frequency, and the sample is substantially transmissive at each of the different frequencies. The method also includes providing a light sensing arrangement to receive light patterns from the incident beams that have passed through the substrate, and analyzing properties of the sample through identifying properties of the light patterns arising from diffraction of the light of the three incident beams by the sample. A difference in frequency between first and second ones of the incident beams is the same as a difference in frequency between second and third ones of the incident beams.

### DESCRIPTION OF INVENTION

This invention relates to an apparatus and method for analysis, and in particular concerns analysis of samples containing small objects such as viruses.

The invention further relates to a sample container. The invention further relates to a cap for a sample container. The invention further relates to a kit of parts comprising a sample container and a kit of parts comprising a cap for a sample container.

The sample container and cap for a sample container are particularly suitable for use in medical testing, virus screening and pathogen screening. The sample container and a cap for a sample container are particularly suitable for use in pathogen screening platforms for testing patients for one or more types of viruses.

There are many circumstances in which it is desirable to be able to detect small objects such as viruses, and also to distinguish reliably between different types of similar objects. For instance, it may be desirable to screen rapidly people who are intending to enter a hospital or other building, or to board a vehicle such as an aircraft.

The spread of COVID-19 in 2020 was made possible in part because individuals could be both contagious and asymptomatic for a period of several days. Such people could look and feel perfectly well, but upon entering a hospital or aircraft could spread the disease to several other people. Such people could not be distinguished from healthy people through asking about symptoms, or by attempting direct measurement of physical symptoms such as body temperature.

Current testing for a condition such as COVID-19 generally involves taking a swab sample and sending the swab to a lab for analysis. At best, results will be obtained within a few days, and clearly this is not a practical way to screen people for entry into a building such as a hospital or care home, or when people are intending to board a vehicle such as an aircraft.

Known sample pots for testing samples typically comprise a transparent container and a removable cap which is screwable onto the container. These have a number of limitations. For example, the pots have to be handled by a clinician and manually opened and closed to access the sample stored inside the container for testing. The sample may easily be spilled or contaminated.

The inventors of the present invention have appreciated a need for an improved sample container that may be more user-friendly and/or which may capable of being handled safely and/or which may be easily and reliably sealable to reduce a risk of spillage or contamination.

It is an objective of the invention to provide an apparatus and method for rapidly and reliably detecting and distinguishing between small objects such as viruses.

Accordingly, one aspect of the present invention provides a cartridge, method of analysing a sample, a light pipe, an analyser and a method of training an analysis system, according to the appended claims and preferred features of the invention.

The term “test plate” is used herein to refer to a component for receiving a patient sample e.g. a smear. It may, but need not, have any specific technical features associated with any known or commercially available types of test plates. It may be a very simple or basic component, for example a substrate made from glass or any other suitable material that may have a circular or rectangular shape, or any other suitable shape that will be apparent to the skilled person.

The invention, in a further aspect, provides a sample container comprising: a body defining an internal cavity, the body comprising a neck portion; a cap engagable with the neck portion so as to close the cavity; and a test plate removably engagable with the body; wherein the cap is configured to hold a swab; wherein the cap is engagable with the neck portion so that a swab is positionable at least partially within the cavity;

and wherein the cap is configured to be slidable along the neck portion so as to adjust a position of a swab within the cavity and relative to the test plate.

The improved sample container or container may be more user-friendly than known sample pots. It may enable self-administration of a patient sample for testing or administration by another user.

When the test plate and cap are engaged with the body, the cavity may be sealed. The cavity may be sealed, for example, to a IP rating of at least IP43, according to the Ingress Protection Code, IEC standard 60529—i.e. protected from water spray less than 60 degrees from vertical. It may also be sealed to a higher IP rating. This may reduce or eliminate a risk of spillage from a sample container. It may also reduce or eliminate a risk of contamination of a sample stored within a sample container. It may also ensure that a clinician does not need to manually open the sample container to access a sample stored within the sample container for testing.

The sample container may be optimised for ease and speed of manufacture. It may be suitable for mass production.

The sample container may be sufficiently robust to withstand manual handling or to operate within a mechanised testing machine.

The neck portion may be an elongate neck portion. It may enable the container to accommodate a swab having a variable length.

Preferably, the neck portion comprises a cap retainer for retaining a cap in engagement with the neck portion. This may for example be an annular ring or enlargement or thickening of the body. This may provide a simple and/or low-cost and/or convenient and/or effective and/or durable and/or easily manufacturable and/or user-friendly means for enabling the cap to be pushed onto the neck portion and for retaining the cap in engagement with the neck portion.

Preferably, the neck portion comprises a cap lock for locking the cap and for opposing further sliding movement of the cap relative to the neck portion. This may, for example be an annular ring or ridge or bump or protrusion or projection. This may provide a simple and/or low-cost and/or convenient and/or effective and/or durable and/or easily manufacturable and/or user-friendly means for locking the cap in a defined position. This may ensure that a swab retained by the cap is stably supported and held out of engagement with the test plate during handling and testing.

Preferably, the cap comprises an annular projection and the cap retainer and cap lock form an annular ring in which the annular projection is receivable so as to opposing further sliding movement of the cap relative to the neck portion. This may provide a simple and/or low-cost and/or convenient and/or effective and/or durable and/or easily manufacturable and/or user-friendly means for locking the cap in a defined position. This may ensure that a swab retained by the cap is effectively supported and held out of engagement with the test plate during handling and testing.

The cap lock may alternatively be, for example, a series of bumps or protrusions, a spline or a separate component engagable with the cap, such as a pin insertable through a hole in the cap and the neck portion, or a C-clip insertable over the neck portion of the sample container between the cap and the body.

Preferably, the cap is configured to be rotatable relative to the neck portion so as to rotate a swab within the cavity and relative to the test plate. This may provide a simple and/or low-cost and/or convenient and/or effective and/or durable and/or easily manufacturable and/or user-friendly means for depositing (i.e. smearing) a sample from a swab retained by the cap onto a surface of the test plate.

Preferably, the cap is slidable towards the test plate and rotatable relative to the test plate so as to bring a swab into temporary engagement with the test plate. This may provide a simple and/or low-cost and/or convenient and/or effective and/or durable and/or easily manufacturable and/or user-friendly means for depositing a sample from a swab retained by the cap onto a surface of the test plate.

Unlike prior art specimen pots/tubes, the cap need not be threaded cap and so need not be screwed and unscrewed to close the cavity.

Preferably, the cap comprises a recess for receiving an end of a swab. This may provide a simple and/or low-cost and/or convenient and/or effective and/or durable and/or easily manufacturable and/or user-friendly means for retaining a swab in engagement with the cap for sliding and/or rotational movement with the cap.

Preferably, the cap recess is at least partially laterally offset from a centreline of the cap. This may provide a simple and/or low-cost and/or convenient and/or effective and/or durable and/or easily manufacturable and/or user-friendly means for securing a swab and depositing a sample on the test plate which has a larger diameter than a diameter of a head of a swab and/or over a larger surface area than a surface area of an end or head of a swab. In other words, it may enable a substantially circular spot of the sample to be deposited on the test plate that has a larger diameter or principal dimension than a width or diameter of a head of a swab. This arrangement may produce an even, circular smear of a predictable size and shape onto the test plate ready for testing.

Preferably, the recess is formed with a plurality of ribs, for example crush ribs, for gripping an end of a swab. This may provide a simple and/or low-cost and/or convenient and/or effective and/or durable and/or easily manufacturable and/or user-friendly means for effectively gripping a swab. It may also provide a simple and/or low-cost and/or convenient and/or effective and/or durable and/or easily manufacturable and/or user-friendly means for ensuring that a swab is locatable on, or substantially parallel to, a principal axis of the cap. The cap may be configured so that a swab is held in such a way that it is positioned on or substantially parallel to a principal axis of the housing and/or on, or substantially parallel to, a centreline of the cavity defined by the housing.

The crush ribs provide flexibility. They may for example enable the cap to accommodate a swab having a shaft with a damaged or deformed end. They may also enable the cap to accommodate a swab having a shaft length that is slightly longer or slightly shorter than a standard or target length of a swab shaft. It may also enable the cap to accommodate a swab having a shaft diameter or width that is slightly larger or slightly smaller than a standard or target diameter or width of a swab shaft.

The recess may alternatively be formed with another means for gripping an end of the shaft. This may, for example, include a series of bumps or protrusions or features of other shapes. Other suitable features will be apparent to the skilled person.

Preferably, a sidewall of the cap is angled to a centreline of the cap. This may facilitate gripping of the cap for movement relative to the neck portion. It may also ensure a more stable coupling between the cap and the neck portion. It may also help to reduce or eliminate any rocking or wobbling of the cap on the neck portion.

Preferably, the cap comprises a first truncated cone shape. This may facilitate gripping of the cap for movement relative to the neck portion. It may also ensure a more stable coupling between the cap and the neck portion. It may also help to reduce or eliminate any rocking or wobbling of the cap on the neck portion.

Preferably, the cap comprises an extension portion or tip. This may facilitate gripping of the cap for movement relative to the neck portion.

Preferably, a swab is receivable within a recess in the cap extension portion. This may provide a simple and/or low-cost and/or convenient and/or effective and/or durable and/or easily manufacturable and/or user-friendly means for effectively gripping an end of a swab. It may also provide a simple and/or low-cost and/or convenient and/or effective and/or durable and/or easily manufacturable and/or user-friendly means for ensuring that a swab is held on, or substantially parallel to, a principal axis of the cap. This may ensure that a swab is held in such a way that, when the body and cap are brought into engagement, a swab is positioned on, or substantially parallel to, a principal axis of the housing and/or on or substantially parallel to a centreline of the cavity defined by the housing.

Preferably, a sidewall of the cap extension portion is angled to a centreline of the cap extension portion. This may facilitate gripping of the cap for movement relative to the neck portion. It may also ensure a more stable coupling between the cap and the neck portion. It may also help to reduce or eliminate any rocking or wobbling of the cap on the neck portion.

Preferably, the cap extension portion comprises a second truncated cone shape. This may facilitate gripping of the cap for movement relative to the neck portion. It may also enable the cap to accommodate a swab with a shaft having a damaged or deformed end. It may also enable the cap to accommodate a swab having a shaft length that is slightly longer or slightly shorter than a standard or target length of a swab shaft.

One of more of the aforementioned features of the cap may provide a “no-wobble” or “anti-wobble” cap retention system. This may ensure reliable, predictable and repeatable contact between a swab retained by the cap and a test plate.

Preferably, the test plate is insertable into an aperture in the body so as to be at least partially located within the cavity.

Preferably, the sample container further comprises a carrier for the test plate.

Preferably, the carrier is insertable into an aperture in the body so as to support at least a portion of the test plate within the cavity. Alternatively, the carrier may be a further cap which holds a test plate and which may be pushed or clipped or screwed or otherwise removably coupled to an end of the body opposite the neck portion to enable a test plate to be removed and accessed for testing.

Preferably, the carrier comprises at least one locking feature. This may ensure a secure engagement with the body. This may provide a simple and/or low-cost and/or convenient and/or effective and/or durable and/or easily manufacturable and/or user-friendly means for ensuring that the carrier is securely engaged with the body. It may also promote sealing between the carrier and the body.

Preferably, the carrier comprises at least one anti-rotation feature. This may be the same as or different to the at least one locking feature. The anti-rotation feature may, for example, be a rectangular tab or projection which may be integrally moulded with the carrier. This may provide a simple and/or low-cost and/or convenient and/or effective and/or durable and/or easily manufacturable and/or user-friendly means for ensuring that the carrier is securely engaged with the body of the sample container and so that the test plate does not move as the cap with retained swab are rotated in engagement with the test plate.

Preferably, the carrier comprises at least one poke yoke feature to ensure accurate engagement with the body. This may be the same as or different to the at least one locking feature and/or the at least one anti-rotation feature. The poke yoke feature may, for example, be a rectangular tab or projection which may be integrally moulded with the carrier. This may provide a simple and/or low-cost and/or convenient and/or effective and/or durable and/or easily manufacturable and/or user-friendly means for ensuring that the carrier is inserted correctly into the sample container.

Preferably, the body is generally tubular. Preferably, the body is closed at an end opposite from the neck portion.

Preferably, the cavity is a substantially cylindrical cavity. This may provide a simple and/or low-cost and/or convenient and/or effective and/or durable and/or easily manufacturable and/or user-friendly sample container body. It may also facilitate sealing of the cavity by the cap for handling and testing. It may also be the most suitable shape to enable rotation of the cap to rotate a swab in contact with the test plate.

Preferably, the body is integrally formed from plastics material. This may provide a simple and/or low-cost and/or convenient and/or effective and/or durable and/or easily manufacturable and/or user-friendly sample container body.

Preferably, the test plate comprises a glass substrate. The glass substrate may have a smooth surface. This may provide a simple and/or low-cost and/or convenient and/or effective and/or durable and/or easily manufacturable and/or user-friendly substrate on which to deposit a sample/smear.

The invention, in a further aspect, provides a cap for sample container comprising: a cap body defining a recess for receiving a swab; wherein the recess is provided with at least one crush rib for gripping a swab.

This may be more user-friendly than known sample pot lids. It may also ensure that a sample container is easily and reliably sealable. In other words, it may seal an end of a sample container, for example, to a rating of at least IP43, according to the Ingress Protection Code, IEC standard 60529—i.e. protected from water spray less than 60 degrees from vertical. It may also be sealable to a higher IP rating. This may reduce or eliminate a risk of spillage from a sample container. It may also reduce or eliminate a risk of contamination of a sample stored within a sample container.

The invention, in a further aspect, provides a kit of parts comprising: a sample container as defined by the invention in the above aspect; and a swab.

This may provide a simple and/or low-cost and/or convenient and/or effective and/or durable and/or easily manufacturable and/or user-friendly means for testing a patient sample.

The invention, in a further aspect, provides a kit of parts comprising: a cap for a sample container as defined by the invention in the above aspect; and a swab. This may provide a simple and/or low-cost and/or convenient and/or effective and/or durable and/or easily manufacturable and/or user-friendly means for testing a patient sample.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

In order that the invention may be more readily understood, embodiments thereof will now be described, by way of example, with reference to the accompanying drawings in which:

FIG. 1 shows a cartridge embodying the present invention;

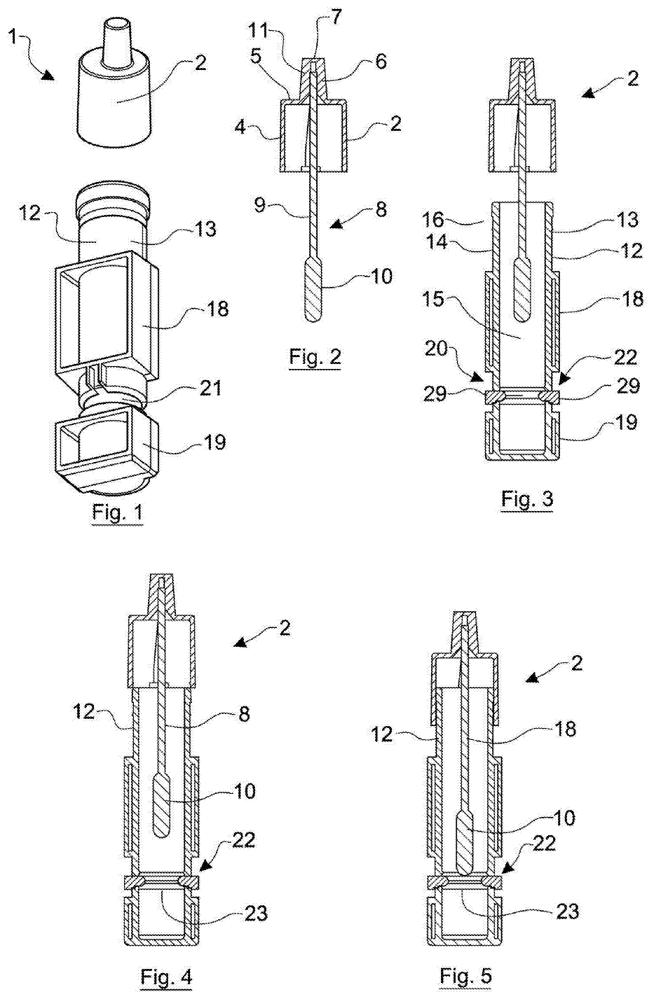

FIGS. 2-6 show stages of use of the cartridge of FIG. 1;

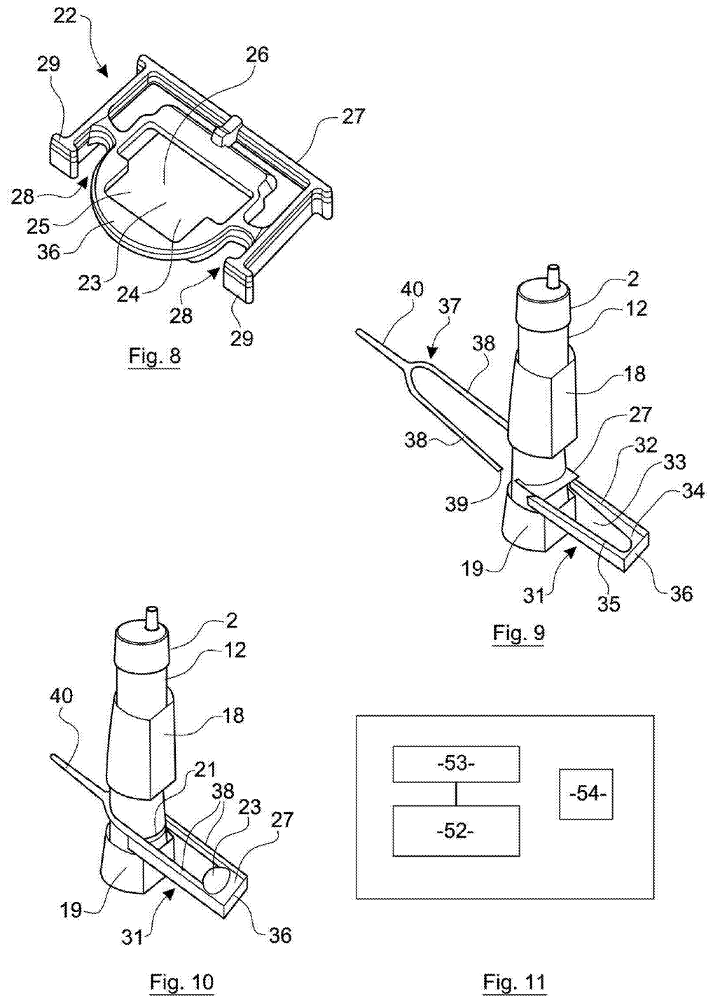

FIGS. 7a to 7c show stages in attachment of a slide carrier to a body of the cartridge;

FIG. 8 shows a slide carrier suitable for use with the invention;

FIGS. 9 and 10 shows stage in the interaction of the cartridge of FIG. 1 with features of an analyser;

FIG. 11 is a diagrammatic representation of an analyser embodying the invention;

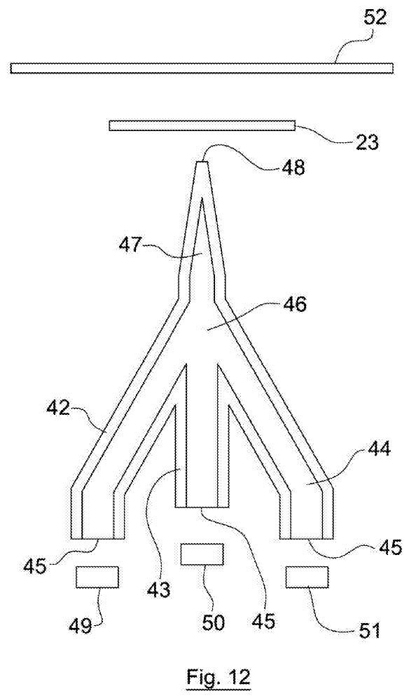

FIG. 12 shows certain components within an analyser embodying the invention;

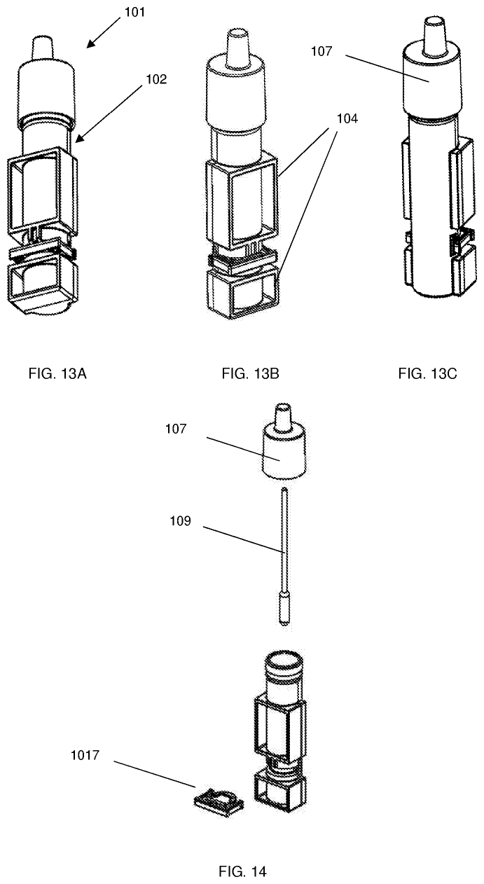

FIGS. 13A-C are a series of views of a sample container embodying the present invention;

FIG. 14 is an exploded view of the sample container of FIGS. 13A-C;

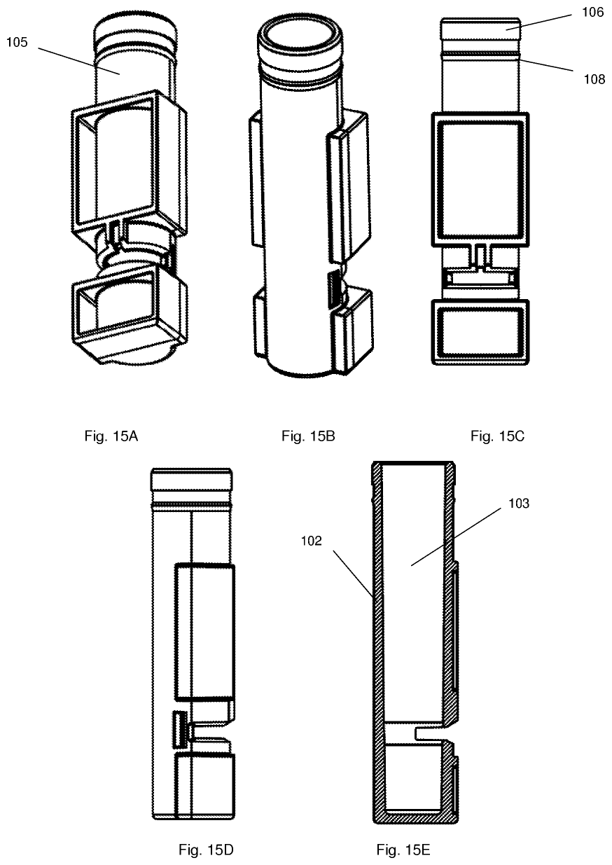

FIGS. 15A-E are a series of views showing the container body of the sample container of FIGS. 13A-C;

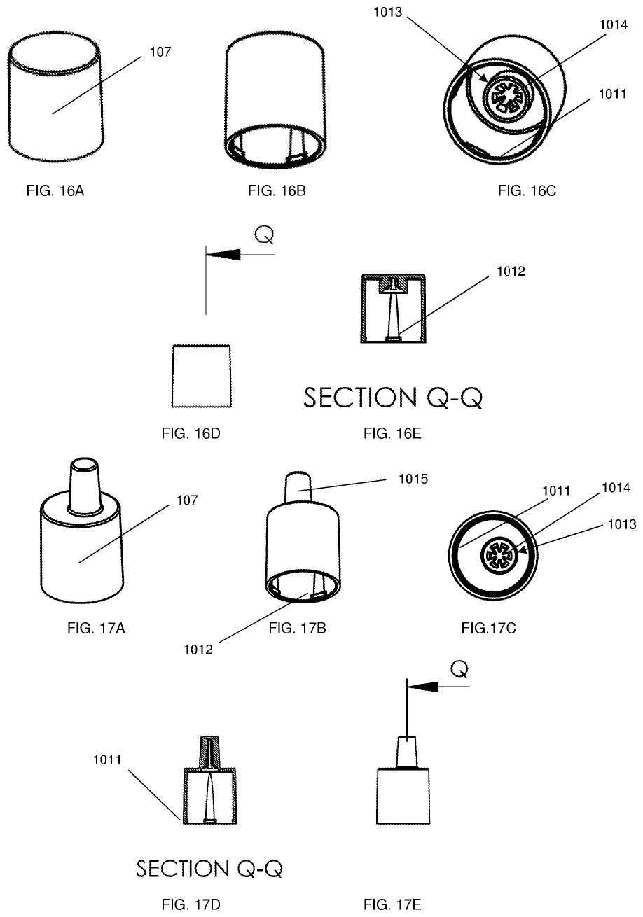

FIGS. 16A-E are a series of views showing a first example cap suitable for use with the sample container of FIGS. 13A-C;

FIGS. 17A-E are a series of views showing a second example cap suitable for use with the sample container of FIGS. 13A-C;

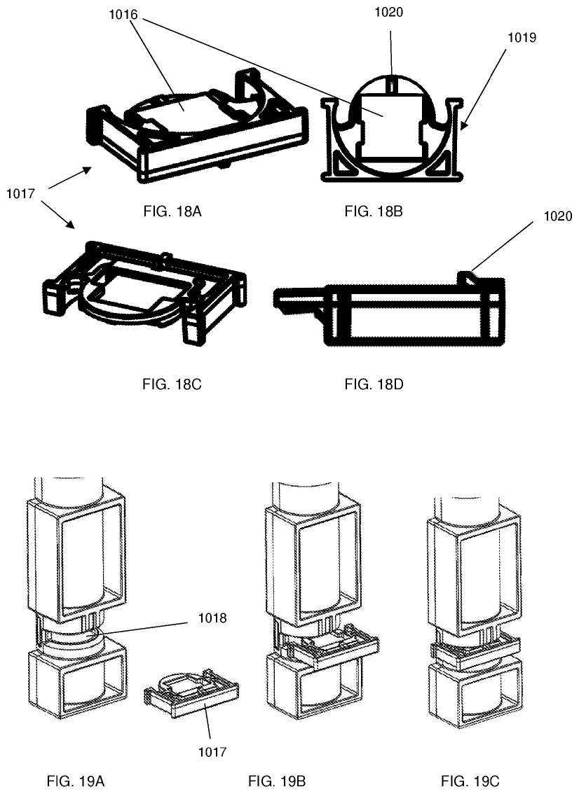

FIGS. 18A-D are a series of views showing a carrier with test plate suitable for use with the sample container of FIGS. 13A-C;

FIGS. 19A-C are a series of views of the sample container of FIGS. 13A-C showing insertion of the carrier;

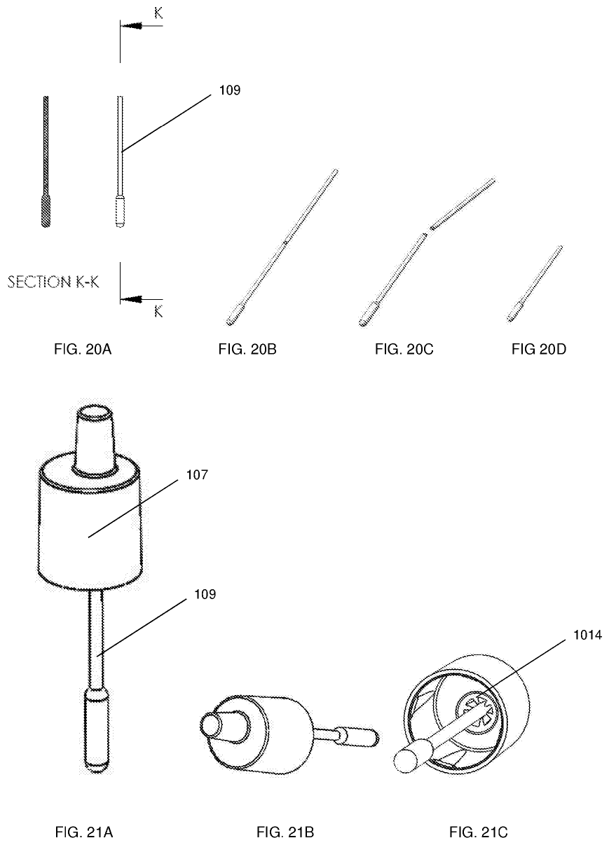

FIGS. 20A-D are a series of views showing a known swab suitable for use with the sample container of FIGS. 13A-C;

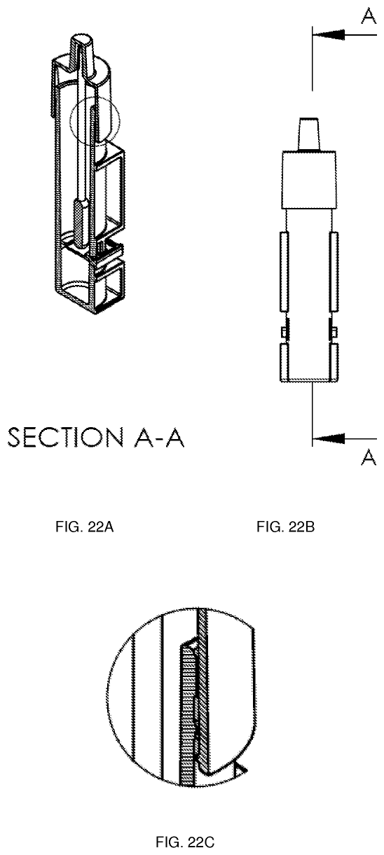

FIGS. 21A-C are a series of views showing a swab of FIGS. 20A-D inserted into the cap of FIGS. 17A-E;

FIGS. 22A-C are a series of section views of the sample container of FIGS. 13A-C;

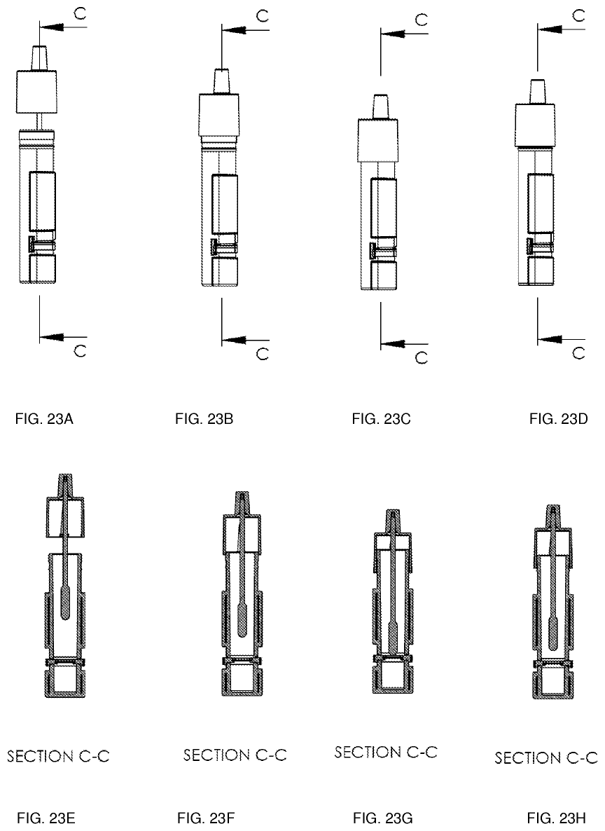

FIGS. 23A-H are a series of views showing the operation of the sample container of FIGS. 13A-C;

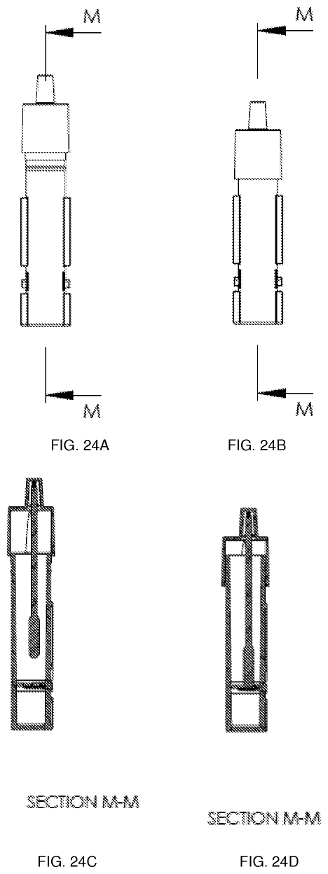

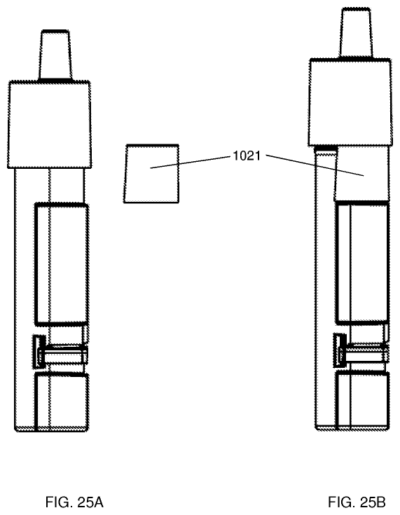

FIGS. 24A-D are a series of section views of the sample container of FIGS. 13A-C; and

FIGS. 25A & B are a series of views show an alternative cap lock for the sample container of FIGS. 13A-C.

Some overlap between the disclosed embodiments will be noted. As mentioned above, and as will be readily appreciated by those skilled in the art, features of different embodiments may be readily combined in any suitable combination. It is to be noted that where different terms are used for like features, these terms may be interchanged, as again will be appreciated by those skilled in the art.

With reference firstly to FIG. 1, a cartridge 1 embodying the present invention is shown. FIG. 1 shows a perspective view of the cartridge 1, and FIGS. 2 to 6 show stages in use of the cartridge 1.

In discussion of the cartridge, terms such as “top”, “bottom”, “upper” and “lower” are used, and it should be understood that these terms refer to the cartridge (and other components which are used with or cooperate with the cartridge) when it is its normal orientation, as shown in the figures. The cartridge may be held or used in any other orientation.

The cartridge 1 includes a lid 2. In FIG. 1 the cartridge 1 and lid 2 are shown separated from each other. The lid 2 takes the form of a generally cylindrical housing having an open lower end 3, a curved side wall 4 and a top side 5. The majority of the top side 5 is generally planar, but a hollow retaining protrusion 6 projects upwardly from the top side 5.

The retaining protrusion 6 is off centre with respect to the longitudinal axis of the lid 2. The cylindrical side wall 4 of the lid 2 has a longitudinal axis, and the retaining protrusion 6 is spaced apart from this axis.

The retaining protrusion 6 is, in example shown in the figures, generally frustoconical, being relatively wide where it meets the planar part of the top side 5, and tapering towards a top end 7 as it rises away from the top side 5. The retaining protrusion 6 is hollow, and the inner walls thereof also taper towards the top end 7 of the retaining protrusion 6. The top end 7 is preferably closed.

The lid 2 is intended to interact with a swab 8. In the example shown in FIG. 2 the swab 8 has an elongate shaft 9, and an adsorbent head 10. The head 10 may be formed from any suitable material, such as cotton, polyester or flocked nylon.

The shaft 9 is primarily provided for the purpose of supporting the head 10, and may be formed from any suitable material, such as wood or plastic.

In use, a relatively long swab may be provided, and once the swab has been used to collect a sample the shaft may be broken at a point along its length. The original, longer shaft may have a narrowed or otherwise weakened section to assist in the breaking of the shaft at the correct point.

The free end 11 of the shaft 9 (i.e. the end furthest from the head 10) may be held in the inside of the retaining projection 6. To do this, the free end 11 is pushed into the interior of the retaining projection 6, where it will be gripped in an interference fit by the frustoconical internal walls. This is the arrangement shown in FIG. 2.

The skilled reader will appreciate that any other method of holding the swab 8 in place with respect to the lid 2 may be used. For instance, the retaining projection 6 may generally cylindrical, rather than frustoconical, and may have one or more flexible ribs formed on its interior which may deform to grip and hold the free end 11 of the shaft 9 of the swab 8.

As mentioned above, the retaining projection 6 is spaced apart from the central axis of the lid 2. This means that a shaft or other tubular object held by the retaining projection will be spaced apart from the central axis of the lid 2.

To begin use of the cartridge 1, a subject may use the swab 8 to obtain a sample, for instance by wiping the swab against the back of his/her throat, and/or wiping the swab against the interior of his/her nostrils.

The shaft 9 of the swab 8 is then attached to the lid 2 as shown in FIG. 2.

Returning to FIG. 1, the cartridge 1 further comprises a body 12. The body 12 has a main cylindrical portion 13, defined by a side wall 14. As can be seen in FIG. 3, the body 12 is hollow, and has an interior space 15. The body 12 is open at its top end 16, and a closed bottom end 17. The outer diameter of the side wall 14 at the top end 16 is approximately the same as, or slightly less than, the inner diameter of the lid 2, so the lid 2 may be placed over the open top end 16 of the body 12.

In preferred embodiments a retaining arrangement is provided so that the lid 2 may be releasably attached to the top end 16 of the body 12. For instance, a protruding rib (not shown) may be formed around the outer side of the side wall 14 of the body 12, near its top end 16, and a corresponding rib (not shown) may be formed on the inner side of the lid 2, near its lower end 3. The dimensions of these ribs are such that, when the lid 2 is placed onto the top end 16 of the body 12, the lid 2 and/or side wall 14 must flex slightly to allow the ribs to pass one another. The skilled reader will appreciate that this will allow the lid 2 to be pushed onto the top end 16 of the body, and the ribs will then retain the lid 2 on the body 12, and prevent the lid 2 from falling off accidentally.

The presence of ribs or a similar retaining arrangement will also provide a click or bump, which will be perceptible to a user, when the lid 2 is correctly placed onto the top end 16 of the body 12, so that the user knows that the lid 2 has been correctly engaged.

In preferred embodiments, when the lid 2 is attached to the body 12, the lid 2 and body 12 together define a generally closed internal space.

In the example shown in the figures the body 12 has upper and lower protrusions 18, 19 which extend away from the outer surface thereof. Each of these protrusions 18, 19 is preferably formed integrally with the side wall 14.

In the example shown in the figures, the upper and lower protrusions 18, 19 each have a cross-sectional shape which is generally rectangular, having a width which is wider than that of the main cylindrical portion 13, and which extends outwardly to one side of the cylindrical portion 13.

In the example shown, each of the upper and lower protrusions 18, 19 extends over a part of the length of the body 12. The upper protrusion 18 is provided around middle of the length of the body 12, and occupies around one third of the total length of the body 12. The lower protrusion 19 occupies around one sixth of the total length of the body 12, and extends near the bottom end 17 of the body 12.

The exact lengths and shapes of the upper and lower protrusions are not essential to the invention. The upper and lower protrusions 18, 19 assist in the ease of handling the cartridge 1, and also ensure that the cartridge 1 is placed into an aperture (discussed below) in the correct orientation.

A slot 21 is formed in the housing 12, and in the example shown the slot 21 is in the region 20 of the body 12 between the upper and lower protrusions 18, 19. The slot 21 is formed through the body 12 substantially perpendicular to the longitudinal axis of the body 12. The slot preferably extends approximately half way through the circumference of the body 12. The slot 21 is preferably formed on the same side of the body 12 as the side on which the upper and lower protrusions 18, 19 extend away from the cylindrical portion 13 of the body 12.

A slide carrier 22 is shown in isolation in FIG. 8. The slide carrier 22 includes a slide portion 23, which takes the form of a planar substrate. The slide portion 23 is preferably of constant thickness. The slide portion 23 is held by a retainer 36, which surrounds the slide portion. The shape and size of the retainer 36 is, in the example shown, such that it fits closely within the hollow internal space of the body 12. In the example shown in the figures the slide portion 23 has a concave upper surface 24, which is highest around its rim 25 and which curves down to a lower region 26 at its centre.

The slide portion 23 is formed from a material which is transparent or substantially transparent at the wavelengths used in analysis (described in more detail below). In preferred embodiments the slide portion 23 is formed from quartz glass, but the invention is not limited to this.

On one side of the retainer 36 is a rim portion 27. The rim portion 27 is preferably of greater thickness than the slide portion 23 and the retainer 36, and extends around approximately one half of the retainer 36. In the example shown in the figures the rim portion 27 has a generally rectangular shape.

The rim portion 27 protrudes away from the retainer 36 in a first direction. The rim portion 27 is also wider than the retainer 36 in a direction generally perpendicular to the first direction.

On either side of the retainer 36, in a direction generally perpendicular with the first direction, the rim portion 27 terminates in a pair of fingers 29. In the example shown the fingers 29 extend in a direction which is generally opposite to the first direction. A gap 28 is formed between the retainer 36 and each of the fingers 29.

In preferred embodiments the retainer 36 and rim portion 27 is formed from a different material to the slide portion 23. The retainer 36 and rim portion 27 may be formed from a plastics material, and in preferred embodiments are integrally formed with each other. The retainer 36 and rim portion 27 do not need to be transparent.

In other embodiments the entire slide carrier 22 may be formed from the same material, such as a transparent plastic. The slide carrier 22 may be moulded or otherwise formed integrally from the material.

Turning to FIG. 3, it can be seen that the slide carrier 22 is engaged in the slot 21 in the body 12.

The retainer 36 and slide portion 23 are received within the slot 21, and the round shape of the retainer 36 generally fills the hollow space within the body 12.

The rim portion 27 is wider than the slot 21, and the rim portion 27 lies outside the body 12, in the region of the slot 21.

The fingers 29 of the rim portion 27 lie on either side of the body 12.

In the orientation shown in FIG. 3, the slot 21 faces generally directly away from the point of view, and the rim portion 27 therefore also faces generally away from the point of view. However, the fingers 29 protrude outwardly beyond the sides of the body 12.

As can be seen from FIG. 1, the overall shape of the rim portion 27 that protrudes from the body 12 when the slide carrier 22 is fully engaged with the body 12 is approximately the same as, or slightly smaller than, the shape of the upper and lower protrusions 18, 19 when viewed along the axis of the body 12.

FIGS. 7a-7c show stages in engaging the slide carrier 12 with the body 12.

When the slide carrier 22 is engaged with the body 12, the side wall 14 of the body 12 is received in the gap 28 between each finger 29 and the retainer 36. There is preferably an interference fit between these components. This helps to retain the slide carrier 22 in place with respect to the body. In preferred embodiments the slide carrier 22 can be repeatedly engaged with, and removed from, the slot 21 in the body 12 without either of these components being damaged.

As can be seen in FIG. 8, each finger 29 protrudes inwardly (i.e. towards the other finger 29) at its far end. The distance between the inner sides of the fingers 29 of the slide carrier 22 is preferably slightly less than the distance between the parts of the body 12 on either side of the slot 21. This means that the fingers 29 must flex as the slide carrier 22 is engaged, and this will also help to ensure that the slide carrier 22 is held firmly in place with respect to the body 12.

FIG. 3-6 show stages in use of the cartridge 1.

As described above, the swab 8 is used to take a sample from a subject, and the swab 8 is then engaged with the retaining protrusion 6 of the lid 2, as shown in FIG. 2.

The lid 2 is then placed onto the open top end 16 of the body 12, as shown in FIGS. 3 and 4, so that the lid 2 is engaged with the body 12 (for instance, in the example described above, by the ribs formed on the lid 2 and body 12 being pushed past each other).

When the lid 2 is first engaged with the body 12, the head 10 of the swab 8 lies above the slide carrier 22.

In the next stage of use, as shown in FIG. 5, the lid 2 is pushed downwardly with respect to the body 12 until the tip of the head 10 of the swab 8 comes into contact with the upper surface 24 of the slide portion 23.

The lid 2 is then rotated with respect to the body 12. Because the shaft 9 of the swab 8 is off-centre with respect to the lid 2, rotating the lid 2 will cause the swab 8 to describe a circular motion within the body 12. The head 10 of the swab 8 will therefore wipe against the upper surface 24 of the slide portion 3 in a circular motion, and the effect of this will be to transfer part of the sample collected by the swab 8 effectively into the upper surface 24 of the slide portion 23.

The concave shape of the upper surface 24 of the slide portion 23 will help to ensure a good contact between the slide portion 23 and the head 10 of the swab 8 as this motion occurs.

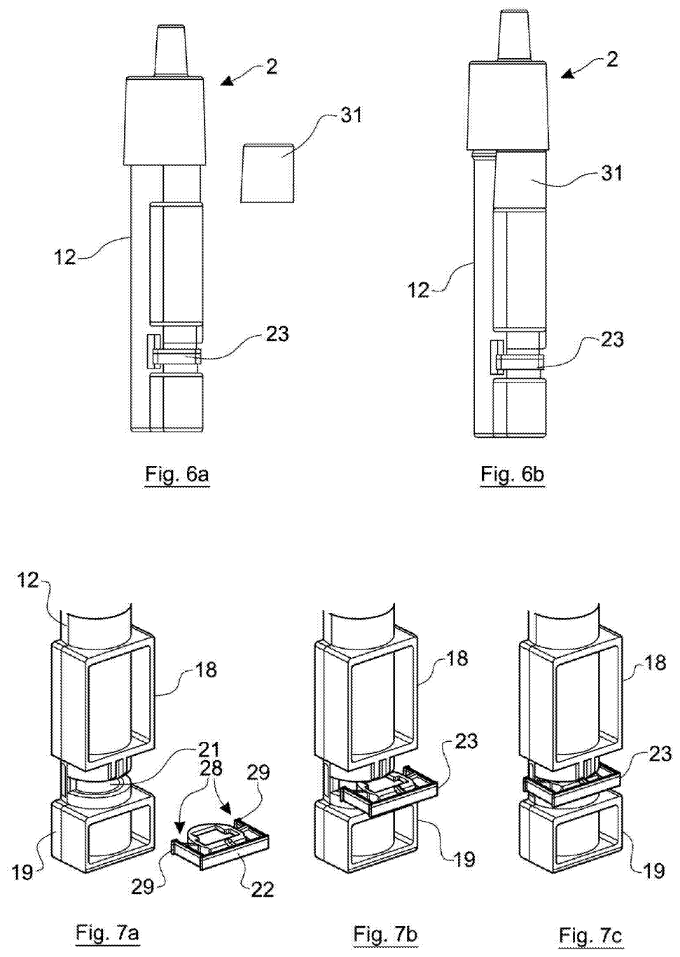

The lid 2 is then lifted upwardly with respect to the body 12, so that the head 10 of the swab 8 is raised above the slide portion 23, and is no longer in contact therewith, as shown in FIG. 4. A collar 31 is provided, as shown in FIGS. 6a and 6b. The collar 31 is generally C-shaped, and has an internal diameter which is approximately the same as, or slightly greater than, the external diameter of the main cylindrical portion 13 of the body 12.

The lid 2 is raised upwardly with respect to the main body 12, and the collar 31 is clipped onto the side of the main body 12, above the level of the upper protrusion 18, and below the lid 2. The collar 31 therefore acts as a spacer between the upper protrusion 18 and the lid 2, and maintains the lid 2 at a certain distance above the upper protrusion 18. In preferred embodiments, when the collar 31 is installed in this way, no part of the swab 8 is in contact with the slide portion 23.

In examples discussed above the lid 2 and body 12 have a cooperating retaining arrangement, such as ribs which are formed on the lid 2 and body 12. In one preferred example, the height 32 of the collar 31 is such that, when the collar 31 is installed, the rib formed on the interior of the lid 2 abuts against the lower side of the rib formed on the outer side of the body 12. This will therefore hold the lid 2 firmly in position with respect to the body 12 when the collar 31 is in place, and will reduce or eliminate “play” or rattling between the lid 2 and the body 12.

The collar 31 is shown installed on the cartridge 1 in FIG. 6b. The cartridge 1 is now ready for use in analysis.

In other embodiments, no collar or other spacer is provided. Instead, the top end 16 of the body 12 and/or the lid 2 may be formed so that, when the lid 2 is attached to the body 12, the lid 2 is biased upwardly with respect to the body 12, so that the lid 2 may be pushed downwardly for the head 10 of the swab 8 to contact the slide portion 23, but when released will rise upwardly to a level at which no part of the swab 8 contacts the slide portion 23. This may be achieved, for instance, by providing cooperating features, which include an inclined plane, on the outer surface of the top end 16 of the body 12, and on the inner side of the lid 2.

In further embodiments, the lid 2 and body 12 include features which allow the lid 2 to be held in a stable manner at a first position, in which the lid 2 is closer to the slide portion 23 (and in which the head 10 of the swab 8 may contact the slide portion 23), and at a second position, in which the lid 2 is further away from the slide portion 23 (and in which the head 10 of the swab may be held away from the slide portion 23). For instance, the inner surface of the lid 2 may include a raised rib or other protrusion, and the outer surface of the top end 16 of the body 12 may have two grooves into which the rib can be received. When the rib is received in a first groove, the lid 2 is in the first position, and when the rib is received in the second groove, the lid 2 is in the second position. The skilled reader will readily appreciate other ways in which this may be achieved.

In a preferred next step, the cartridge 1 is inserted into an analyser. The analyser preferably has an entry aperture (not shown) which is shaped so that the cartridge 1 can only be inserted into the entry aperture in the correct rotational orientation. The skilled reader will understand that the presence of the lower protrusion 19 at the lower end of the body 12 means that the cross-sectional shape of the body 12 is asymmetrical.

Within the analyser the cartridge comes to rest (through dropping under the influence of gravity, or by any other means) in an analysis position. A carriage 31 is positioned within the analyser, level or approximately level with the slide carrier 22. As can be seen in FIG. 9, the carriage 31 has a tray region 32 having a central aperture 33, and a rim 34 having an upward-facing receiving surface 35. An end of the carriage 31 furthest from the cartridge 1 has an end wall 36, whose shape generally matches at least a part of the shape of the rim portion 27 of the slide carrier 22.

The carriage 31 is moveable within the analyser in a generally horizontal direction, preferably by means of a stepper motor or other similar drive arrangement. The carriage 31 is preferably movable in a direction which is substantially generally towards and away from the cartridge 1, when the cartridge 1 is in its analysis position.

A pusher 37 is provided on the opposite side of the cartridge 1 from the carriage 31. The pusher 37 has a pair of pushing fingers 38, which are preferably generally parallel and spaced apart from one another. The spacing of the free ends 39 of the pushing fingers generally matches the distance between the fingers 29 of the slide holder 22.

The example shown, the pusher 37 has a main stem 40, to which the two pushing fingers 38 are attached, and in the example shown in the figures the pusher 37 has a shape resembling that of a tuning fork. However, this shape is not essential, and it is also envisaged that the pushing arrangement may comprise two separate pushing members, which are not connected to each other.

Once the cartridge 1 has arrived in its analysis position, the pusher 37 may be advanced so that the free ends 39 of the pushing fingers 38 contact the fingers 29 of the slide carrier 22. The pusher 37 is advanced further so that the slide carrier 22 is disengaged fully from the body 12 of the cartridge 1, and is pushed onto the carriage 31. The pushing action continues until the rim portion 27 of the slide carrier 22 contacts the end wall 36 of the carriage 31, as shown in FIG. 10. At this point, the slide carrier 22 is preferably completely removed from the carriage 1, and is in its analysis position.

In this analysis position, at least a part of the slide portion 23 of the slide carrier 22 is positioned over the aperture 33 formed through the carriage 31. The slide carrier 22 is held in place by its side regions resting on the side surfaces 34 of the tray 32.

With reference to FIG. 12, the slide portion 23 of the slide carrier 22 is shown, removed from the cartridge 1, and positioned for analysis. Features of the slide carrier 22 other than the slide portion 23, and also features of the carriage 31, are not shown for the purposes of clarity.

A method for analysis of the swab sample is described below, and it should be understood that this method is separate from the features of the cartridge, and its interaction with the analyser, that are described above. The cartridge is a convenient and effective way of gathering a sample from a subject, transferring the sample to a slide carrier, and positioning the slide carrier for analysis. However, the analysis method described below could equally be used for analysing a sample that is gathered and/or prepared in any other way. The cartridge could also be used for gathering samples for use in other types of analysis.

A light pipe 41 is positioned on one side of the slide portion 23. In the example shown in FIG. 10 the light pipe 41 is positioned below the slide portion 23, although this is not essential.

The light pipe 41 has three entry guides 42, 43, 44. Each entry guide has a receiving end 45, through which light may enter the entry guide 42, 43, 44. The receiving ends of the three entry guides 42, 43, 44 are separate from one another, and are preferably spaced apart.

The three entry guides 42, 43, 44 extend from their receiving ends 45 and converge on a junction 46. An exit guide 47 extends from the junction, and has an emitting end 48 through which light may leave the light pipe 41.

The design of the light pipe 41 is such that light may enter the receiving end 45 of any one of the three entry guides 42, 43, 44, be transmitted along the respective entry guide 42, 43, 44, through the junction 46 and then along the exit guide 47 and out of the light pipe 42 through the emitting end 48.

Importantly, in preferred embodiments light which enters the light pipe 41 through the receiving end 45 of any one of the three entry guides 42, 43, 44 will leave the emitting end 48 of the light pipe 41 along exactly or substantially the same axis.

In the embodiment shown in the figures the exit guide 47 tapers from the junction 46 to the emitting end 48, so that light travelling along the exit guide is channeled to a relatively small area, thus helping to ensure that light leaving the light pipe 41 through the emitting end 48 always travels along the same or substantially the same axis.

In preferred embodiments, the width of each entry guide 42, 43, 44 is substantially constant along its length. The width/cross-sectional area of each entry guide is preferably chosen to match as closely as possible the width of the beam that will enter each entry guide 42, 43, 44. The exit guide 47 preferably tapers from the junction 46 to the emitting end 48, and the width of the exit point at the emitting end 48 preferably functions as a pinhole, for the wavelengths of light that are used. In examples of the invention the diameter of the emitting end may be in the range of 20-50 μm.

The exit guide 47 preferably tapers consistently from the junction 46 to the emitting end 48, and has straight or substantially straight sides over the tapering portion thereof.

In the example shown in the figures, each entry guide 42, 43, 44, and the exit guide 47, are each generally hexagonal in cross-sectional shape. However, this is not essential, and any suitable cross-sectional shape may be used.

As the skilled person will appreciate, the main internal structure of the light pipe 41 is formed from a transparent material, and preferred embodiments of the invention use acrylic (PMMA) as this material. However, any other plastic or other suitable material may be used for the main internal transmitting structure of the light pipe 41.

The transmitting structure of the light pipe 41 is preferably coated with a reflective material, such as aluminium.

It is also preferred that the outer surface of the light pipe 41 (outside the reflective material, if one is provided) is coated with a black or otherwise light-absorbing paint or other covering.

As the skilled reader will appreciate, a light pipe of this type will operate through total internal reflection (TIR) of light travelling along the length of the light pipe 41.

The addition of a reflective coating will help to ensure that any photons which are not totally internally reflected by the main optical material of the light pipe 41 will be reflected inwardly and remain within the light pipe 41. The outermost light-absorbent coating will help to ensure that no photons enter the light pipe 41 from outside.

The light pipe 41 is preferably a substantially rigid structure, with the spatial arrangement of the entry guides 42, 43, 44 and the exit guide 47 being substantially fixed. The light pipe 41 is preferably different from a collection or arrangement of optical fibres.

Returning to FIG. 12, the light pipe 41 is arranged so that the emitting end 48 thereof points at a central region of the slide portion 23, so that light leaving the emitting end 48 of the light pipe 41 will be transmitted through the central region of the slide portion 23.

First, second and third LEDs 49, 50, 51 are positioned adjacent the receiving ends 45 of the first, second and third entry guides 42, 43, 44 respectively. Each LED 49, 50, 51 is arranged so that light generated thereby is emitted into the receiving end 45 of the respective entry guide 42, 43, 44.

The skilled reader will understand from the foregoing that light emitted by any one of the three LEDs 49, 50, 51 will be transmitted along the light pipe 41, and leave the light pipe 41 through the emitting end 48 thereof, passing through the slide portion 23.

As mentioned above, light generated by each of the three LEDs preferably exits the light pipe 41 along the same or substantially the same axis. This has been found to be important for reliable analysis, and the inventors have found that small differences in alignment can lead to adverse effects. The axes along which the beams exit the emitting end 48 of the light pipe 41 should the same, or as closely aligned as possible. In preferred embodiments there is a deviation of less than 1° between beams, and more preferably a deviation of less than 0.1° between beams.

In preferred embodiments of the invention the light emitted by each of the three LEDs 49, 50, 51 is optical or ultraviolet (UV) light. The three LEDs 49, 50, 51 emit light at different frequencies.

In one embodiment, the LEDs 49, 50, 51 emit light having wavelengths of exactly or approximately 425, 475 and 525 nm, respectively.

It is not essential that the sources of incident light are LEDs. Any other suitable light sources, which emit light at a defined frequency, may be used.

According to the inventors' present understanding, the exact frequencies used are not essential. However, it is important that the three frequencies are different from one another. The inventors have found that the difference between the wavelengths can be as little as 1 nm. It is preferred that the spacings between the frequencies are equal or substantially equal (for instance, in the above example, the spacing between each wavelength is 50 nm).

If any of the wavelengths used are above around 600 nm, it is likely that this will lead to fluorescing from the sample on the slide portion 23. Wavelengths above 600 nm are therefore not preferred.

Wavelengths below 50 nm are also not preferred. One reason for this is that the analysis relies on diffractive effects, which may be lost or diminished at such frequencies.

Therefore, in preferred examples of the invention, the three LEDs 49, 50, 51 emit light at wavelengths which are spaced apart from one another, and are all between 50 and 600 nm. As discussed above the spacing between the wavelengths is preferably the same or approximately the same.

On the other side of the slide portion 23 from the light pipe 41 is a light sensing arrangement 52. The light sensing arrangement 52 preferably takes the form of a CCD array. It has been found that examples of the invention work well with a 13 megapixel sensor, although it is anticipated that any CCD array having a dot pitch of 1.2 μm of less will also produce acceptable results. Example systems used by the inventors have employed CCD arrays similar to those used in the cameras of mobile phones.

In preferred embodiments, a 60 megapixels or greater CCD array may be used, or a CCD array having a dot pitch of 0.8 μm.

The light sensing arrangement 52 preferably detects both the amplitude and wavelength of light which falls on each of its sensing elements (pixels, in the case of a CCD array).

Clearly, it is important that the light sensing arrangement 52 is reliably able to detect light of the frequencies which are emitted by the three LEDs 49, 50, 51.

In an analysis method embodying the invention, light is emitted from the three LEDs 49, 50, 51 in turn. Light may be emitted from each of the LEDs 49, 50, 51 for a period which is sufficient for the light sensing arrangement 52 to register fully an image from light which originates from each LED 49, 50, 51, and this may be informed by the integration time of the light sensing arrangement 52. In examples of systems used by the inventors, each LED 49, 50, 51 emits light for a period of 800 ms, although it is envisaged that the emission time may be 400-500 ms or less. As light is emitted by each LED 49, 50, 51 it will be transmitted through the light pipe 41, pass through the slide portion 23, and be detected by the light sensing arrangement 52. Information from the light sensing arrangement 52 is transmitted to a processing arrangement (not shown).

As light is emitted from the light pipe 41 and through the slide portion 23, the light will be diffracted as it passes the sample left on the surface of the slide portion 23 by the swab 8. As the skilled reader will appreciate, this will cause deflection of the light as it passes through the sample.

Without wishing to be bound by theory, the inventors' current understanding is that light in each incident beam interacts with electrons (i.e. an electron cloud) at the outer edge of a virus which is present in the sample. Because the wavelength of the light used is larger than the size of the cloud, diffraction of the light will take place, giving rise to interference patterns in the resulting light which impinges on the light sensing arrangement. It is expected that these effects will be diminished for incident wavelengths below 50 nm.

The degree of deflection caused by the diffraction effects will depend upon the wavelength of the light. The level of diffraction will therefore differ for the light emitted by each of the three LEDs 49, 50, 51.

As light is emitted from each of the three LEDs 49, 50, 51, a light pattern or signature will be detected by the light sensing arrangement 52. Due to the different levels of diffraction arising from the different wavelengths emitted by the LEDs 49, 50, 51, the light pattern impinging on the light sensing arrangement 52 will be different for each LED 49, 50, 51.

It has been found that, using three frequencies of UV light in this way produced light patterns that can be used to distinguish between different viruses which are present in the sample on the slide portion 23. In other words, if a particular virus is present in the sample, this will give rise to a series of light patterns that are sufficiently different from the light patterns produced by other viruses that, based on the light patterns alone, it is possible to distinguish between different viruses that may be present in the sample.

In the discussion above it is mention that it is preferred that the spacing between the three frequencies emitted by the LEDs 49, 50, 51 are the same or substantially the same. A benefit of this is that the light patterns produced by the three LEDs will contain consistent differences (e.g. a difference between the pattern produced by the light from the first LED 49 and the pattern produced by the light from the second LED 50 will be similar to a corresponding difference between the pattern produced by the light from the second LED 50 and the pattern produced by the light from the third LED 51).

In preferred examples of the invention, light patterns received by the light sensing arrangement are analysed by a semantic scene segmenting network, which the skilled reader will understand is an example of an artificial intelligence (AI) image classification algorithm. The skilled reader will be aware of how to implement a semantic scene segmenting network for an application of this type. As an example, convolutional artificial neural networks are commonly used to achieve semantic scene analysis of this type. Examples of suitable semantic scene segmenting networks that could be used for this purpose are VGG Oxford (https://www.robots.ox.ac.uk/˜vgg/) and SegNet (https://mi.eng.cam.ac.uk/projects/segnet/).

To prepare the semantic scene segmenting network for use in a method embodying the invention, the network is preferably trained on suitable examples. In one example carried out by the inventors, a network was trained through exposure to samples of six different types of viruses: SRI (serious respiratory illness), 2RS (respiratory syncytial virus), Influenza A, 22NE (common cold), Adeno virus, and SARS-CoV 2 (i.e. COVID 19) and SARS 1 (which has identical surface proteins). Samples of each virus were applied to the surface of a slide or other suitable substrate. Light of the same frequencies as emitted by the three LEDs 49, 50, 51 was then transmitted through the substrate, so that it impinged upon a light sensing arrangement. The resulting light patterns were provided to the semantic scene segmenting network, and associated with the correct virus type. As the skilled reader will understand, upon being provided with a sufficient number of training examples, the network will then be able to categorise a new sequence of light patterns as being associated with a particular one of the six viruses, and distinguish this from any of the other six viruses.

In the example carried out by the inventors, the semantic scene segmenting network was trained through exposure to light patterns produced by light passing through substrates on which samples having known viruses were deposited. The skilled reader will understand how training data of this kind can be provided to an image recognition algorithm in order to train the algorithm suitably.

The semantic scene segmenting network is then trained to recognise distinctive features in light patterns which arise from the presence of each of the viruses on which the network has been trained. In preferred embodiments, upon receiving a new set of light patterns, the network produces a different semantic map for each class of object that is to be recognised (i.e. for each different virus). Within each map, the network will identify features which indicate that one instance of the virus (e.g. one virion) is present in the sample.

In preferred embodiments, a threshold is set for the number of detected instances that will give rise, in each class, to a positive determination that that class of object (e.g. that particular virus) is present in the sample. In one example, the threshold for producing a positive determination that a sample contains coronavirus is 10. In other words, if the network determines that there are 10 instances of the virus in the sample, a positive determination is given. If 9 or fewer instances are detected, a negative determination is given.

Different thresholds may be set for each class of object, and these thresholds may be set in a way that takes into account the ease with which instances of the object are detected, the level of certainty that is required, and so on. The skilled reader will be aware of how to set appropriate thresholds for a system of this kind.

The semantic maps are analysed separately, and a separate determination is therefore reached as to whether each of the classes of objects is present (or present in a number that exceeds a relevant threshold). This means that the system can give an output indicating that the subject has none of the viruses in question, that the subject has a particular one of the viruses in question, or indeed that the subject has two or more identified ones of the viruses.

As the network is trained to recognise further viruses, this can be added to the analysis that is carried out by the network, and so the number of viruses that can be identified (in isolation, or in combination with any others of the viruses) can be increased without sacrificing the reliability of the system in identifying any particular virus of interest.

The skilled reader will understand that, since there is no objective lens, the light sensing arrangement 52 does not receive a focused image, but instead captures the light field falling thereon. This light field effectively comprises a multi-dimensional hologram. Light is captured at all (or a wide range of) focal lengths. The image recognition algorithm will be able to resolve the impinging light into relevant information, and in preferred examples the algorithm will find or determine an optimal focal length for each aspect of the analysis.

In preferred examples of the invention, the light patterns output by the light sensing arrangement 52 are analysed locally, by one or more processors 53 within the analyser. In an example, the semantic scene segmenting network is implemented in a field programmable gate array (FPGA) device, and the skilled reader will readily appreciate which FPGA devices will be suitable, although the invention is not limited to this. Any suitable combination of hardware and software (including accelerated computing devices) may be used to implement the processing of the method. FIG. 11 shows schematically some of the components of an analyser embodying the invention.

If the processing of the method is carried out locally, this will avoid any problems associated with loss of transmission between the analyser and an external processing location. Local processing also avoids legal or regulatory complications that may arise through transmission of personal and/or medical data. However, it is envisaged that in some settings the analyser may transmit data including or representing some or all of the light patterns output by the light sensing arrangement, via a transmitter 54, and transmit them to a remote location for processing.

In examples carried out by the inventors, processing of the light patterns output by the light sensing arrangement takes around 5-20 seconds. The analyser preferably includes a screen or other output device which can display the result of the analysis. This output may simply take the form of a positive or negative indication, but may be more detailed than this, depending on the requirements of any particular situation.

The skilled reader will understand that the apparatus and method disclosed above allow a subject to take a swab sample easily, and for this sample to be placed in an analyser, which will then swiftly produce an output which indicates whether the subject has a particular virus, such as coronavirus (i.e. SARS-CoV-2). The entire process may take around 30 to 60 seconds, and is sufficiently fast to be used, for example, in a queue to board an aircraft. This technique will therefore be valuable in helping to halt the spread of COVID 19, while allowing travel by air, train etc. to continue. The skilled reader will also see that this method can be rapidly adapted to screen for new viruses which appear, and thus could be used at an early stage of a new epidemic, or potential epidemic, which is based on a virus.

Returning to the method, in preferred embodiments the slide carrier 23 and/or cartridge 1 are ejected from the analyser into a waste bin or other collection vessel. The analyser will then be ready to receive a new cartridge for analysis of a further sample.

In preferred embodiments, the carriage 31 is moved towards the cartridge 1, so that the slide carrier 23 is re-engaged with the slot 21 formed in the body 12. The cartridge 1 is then released from its analysis position and falls into a waste bin. The skilled reader will appreciate that there are many ways in which this may be achieved. It is also envisaged that the cartridge 1 and slide carrier 23 may be ejected from the analyser separately, and may fall into different collection vessels.

The invention is not limited to detecting and distinguishing between different viruses, and may also find utility in detecting and distinguishing between any other types of three-dimensional object having a size up to around 800 μm.

In the examples discussed above, three different frequencies of light are used. The inventors have found that this number of different frequencies allows the network to be able to distinguish between different viruses with sufficient reliability. More different frequencies (for examples, four, five, six or more) could be used, and this would increase the sensitivity of the technique. However, since the inventors have found three frequencies to be sufficient, this is used in the examples to avoid excessive cost. It is envisaged that use of more than three frequencies may be desirable in certain applications.

The inventors also envisage that the invention may be carried out using only two frequencies of light. It is important that at least two are used, because use of only one light source will not provide the spectral information that is needed for the analysis to be carried out. However, a system with only two frequencies of light, along with the other components described above, will be able to distinguish reliably between different viruses. The skilled reader will be aware of how the examples described above can be adapted for use with two light sources.

In the examples discussed above, the three incident beams of light are transmitted through the slide carrier sequentially. However, the invention has also been found to work if the incident beams are transmitted through the slide carrier simultaneously. The resulting combined light pattern contains sufficient information that a suitably trained image recognition algorithm (such as a semantic scene segmenting network) can reliably distinguish between different viruses which are present in a sample on the slide carrier.

The inventors have found that a technique as described above, including a 13 megapixel CCD array and with a semantic scene segmenting network implemented on an FPGA device, has a sensitivity of 99.8%, and a specificity of 96.7%, for the six viruses on which the apparatus was trained.

With reference to FIGS. 13A to 14, a sample container 101 is provided. The sample container may provide a user-friendly and/or self-administrable and/or auto-smearing sealable container which can be handled and/or disposed of in a convenient safe manner.

In the example embodiment shown in the various Figures, the sample container is a test cartridge or sample tube. The cartridge is suitable for insertion into a suitable testing machine for testing a patient sample stored within the cartridge.

In other embodiments, the sample container may be a standalone sample container for storing a patient sample which can then be tested in a known way, which may involve removing the sample and testing it in a laboratory or other testing environment using one or more known techniques.

As shown in FIGS. 15A to 15E, the test container has a generally tubular body 102. The body is hollow so as to define an internal cavity 103. The cavity is a substantially cylindrical cavity.

The container body may be provided with an external shape and configuration that may enable it to be easily gripped by a user. Where the container is for use with an automated testing machine, the container body may be provided with an external shape and configuration that enables the container to be inserted into a correspondingly-shaped aperture or dock in a test machine. In particular, the body may be provided with one or more protrusions or poke yoke features 104 to ensure that the container is insertable into the machine in only the correct positon and/or orientation.

The container body may also be provided with one or identifiable elements, identification features or traceability features (not shown) to ensure that the container is of an appropriate type and is compatible with the testing machine.

The body has a neck portion 105. The neck portion is hollow and forms part of, or an extension of, or leads to, the cavity. The neck portion is provided with a cap retainer 106 for retaining a cap 107. An outer end of the cap retainer is provided with a chamfered or curved edge to facilitate positioning of a cap over the neck portion. An inner end of the cap retainer is provided with a step or shoulder to resist removal of the cap from the neck portion.

The neck portion is also provided with a cap lock 108 for locking a cap i.e. opposing further sliding movement of the cap relative to the neck portion.

A cap or lid is shown in FIGS. 17A to 17E. The cap is engagable with the neck portion so as to close the internal cavity. As will be described further below, the cap is configured to hold a swab 109. The cap is engagable with the neck portion so that a swab is positionable within the cavity.

The cap is configured to be slidable along the neck portion so as to adjust a position of a swab within the cavity and relative to a test plate.

The cap comprises an annular ridge or bump or projection or narrowing 1011. The cap retainer and cap lock form an annular gap or ring or recess in which the annular projection is receivable so as to oppose further sliding movement of the cap relative to the neck portion.

The cap comprises one or more stability enhancing features 1012 on an inside surface of the cap sidewall. These may promote a stable sliding engagement of the cap relative to the neck portion and may serve to minimise or eliminate rocking or wobbling of the cap.

In addition to being slidable relative to or along the neck portion, the cap is also rotatable relative to the neck portion so as to rotate a swab within the cavity and relative to the test plate. As such, the cap is slidable towards the test plate and rotatable relative to the test plate so as to bring a swab into temporary engagement with the test plate and deposit a sample or smear onto the test plate.

The cap comprises a recess 1013 for receiving a swab. The recess is at least partially laterally offset from a centreline of the cap. The recess is formed with a plurality of projections or crush ribs 1014 for gripping a swab. A preferred number of crush ribs is six. These are spaced evenly around a perimeter of the recess.

A sidewall of the cap is angled to a centreline of the cap. In other words, the cap generally assumes a first truncated cone shape.

The cap comprises an extension or tip portion 1015 to facilitate gripping and/or rotation of the cap. The extension portion is at least partially offset relative to the cap. In other words, the extension portion is at least partially closer to one side of the cap than to an opposite side, or the extension portion is at least partially laterally offset from a centreline of the cap and/or a centreline of the container body and/or the cavity. The cap and or the extension portion may have, for example, a ribbed or knurled surface (not shown) to facilitate gripping by a user. A swab is insertable into the cap and embeddable within the cap extension portion.

A sidewall of the cap extension portion is angled to a centreline of the cap extension portion. In other words, the extension portion generally assumes a second truncated cone shape. The crush ribs are also angled to a centreline of the cap extension portion. In other words, a swab-receiving aperture defined between the ribs may be wider at an inner end where the extension portion joins the cap that at its other outer end. This serves to control the proportion of a length of a swab shaft that is insertable into the aperture of the extension portion i.e. the further a swab shaft is advanced into the recess, the harder it gets to further insert it.

The cap and the container body may be provided with a visual marking (not shown), for example lines or arrows or bumps or other projections, to enable a user to track rotation of the cap relative to the neck portion.

As shown in FIGS. 18A to 18D, the container is provided with a test plate 1016. The test plate is provided in or on a carrier 1017. The carrier is insertable into an aperture in the container body so that at least part of the test plate extends into the cavity and is generally aligned with the neck portion. In the embodiments shown in the Figures, the carrier is insertable into a correspondingly-shaped aperture 1018 in the body to advance the test plate into the cavity. The carrier with test plate is removable from the container for testing of a patient sample.

The test plate is a glass substrate for receiving a patient sample through contact with a swab insertable into the container. The test plate has a smooth surface for receiving a sample. The test plate may be between about 0.5 mm and about 1.0 mm thick, preferably about 0.8 mm think. The test plate may be pushed or clipped or slid into a correspondingly shaped recess in the carrier. Optionally, it may also be secured in the carrier using an appropriate retaining means, such as an adhesive or one or more retaining clips.

The carrier is provided with at least one locking feature 1019. This helps to ensure a secure engagement between the carrier and the body. This may comprise one or more clips, which may be sprung clips, for engagement with one or more corresponding recesses in the wall of the container body. These may ensure that the body is sealed to prevent contamination or leakage of the sample from the cavity. The carrier is removable from the body, either using manual force sufficient to overcome the locking feature(s), or in an automated manner when the container is inserted into a testing machine. Removal of the carrier may require one or more sprung clips to be moved or squeezed together. Other suitable locking features will be envisaged by the skilled person. For example, the locking feature may simply be a tight or interference fit between the carrier and the body, or channels along which the carrier slides and has a frictional fit.

The carrier is provided with at least one anti-rotation feature 1020. This may be the same as or different to the at least one locking feature. The anti-rotation feature may, for example, be a rectangular tab or projection which may be integrally moulded with the carrier. Other suitable anti-rotation features will be envisaged by the skilled person

The carrier is shaped in non-uniform way, or is provided with at least one poke yoke feature, so as to ensure accurate engagement with the body. This may be the same as or different to the at least one locking feature and/or the at least one anti-rotation feature. For example, the carrier may be shaped in such a way that it can only be inserted in to the container body in the correct orientation. Alternatively the carrier may comprises a poke yoke feature, for example, a rectangular tab or projection which may be integrally moulded with the carrier. Other suitable poke yoke features will be envisaged by the skilled person.

The container is usable in conjunction with a swab 109. A suitable swab may be a known type of swab comprising a shaft and a head. A head and upper part of a swab shaft may be separable from a lower part of a swab shaft to reduce a length of a swab. A swab shaft may be provided with one or more break points or snap points. A suitable swab may have a length of about 150 mm. It may have a predefined break point on a shaft to reduce the length of the operative part of a swab to about 78 mm or about 79 mm or about 80 mm. A diameter of a shaft of a swab may be about 2 mm to about 3 mm.

The container body is formed from plastics material. A suitable material is polycarbonate, preferably hard polycarbonate. The container body is integrally moulded with any poke yoke features required for compatibility with a testing machine. Other suitable materials will be known to the skilled person.

The cap is also formed from plastics material. A suitable material is polycarbonate, preferably hard polycarbonate. Other suitable materials will be known to the skilled person.

The cap is integrally moulded with the aperture and crush ribs for receiving and securely supporting a swab.

The carrier is formed from plastics material. A suitable material is polycarbonate, preferably hard polycarbonate. Other suitable materials will be known to the skilled person.

The test plate is made from glass. Other suitable materials will be known to the skilled person.

A swab useable with the container may be a known type of swab made from plastics material. Suitable materials include polycarbonate for the shaft and a cotton or polyester material for the head. Other suitable materials will be known to the skilled person.

A first kit of parts may be provided which comprises a container as described above and at least one swab of a known type.

A second kit of parts may be provided which comprises a cap as described above and at least one swab of a known type.

In operation of the test container, as shown by the various Figures, in particular FIGS. 19A to 24D, a patient or other user inserts a carrier 1017 with test plate 1016 into the correspondingly-shaped aperture 1018 in the container body 102. A swab 109 is used to collect a patient sample. A swab is then snapped at a break point to leave a shorter, operative part of a swab. An operative part of a swab is then insertable into the recess 1013 in the cap 107. The crush ribs 1014 are configured to hold a swab in a secure manner and so as to emerge from the cap substantially orthogonally from the cap and/or substantially parallel to a centreline of the cap.

The patient or other user grips the extension portion 1015 of the cap 107 and pushes the open end of the cap onto the neck portion 105 of the body 102, the annular projection 1011 of the cap being pushed over the cap retainer, thereby positioning a swab inside the neck portion of the container. The cap is advanced over the cap lock 108 and further towards the body to advance a swab along the neck portion and into the cavity 103 towards the test plate 1010. The cap is further advanced along the neck portion so that a swab comprising the patient sample is brought into contact with the test plate.

While maintaining sufficient force to ensure continued contact between a retained swab and the test plate, the cap is rotated relative to the neck portion by turning the extension portion. The cap is preferably rotated through one or two or three or four or five compete revolutions of the cap to deposit a portion or smear of the patient sample on the test plate. A particularly preferred number of revolutions is three. The number of revolutions may be monitored by the user looking at the visual markings on the cap and/or neck portion. Due to the offset of the extension portion, rotation of the cap deposits a sample or smear on the test plate which covers a greater surface area than a head of a swab.

The cap is then retracted along the neck portion until the annular projection on the cap is received within an annular space or recess defined between the cap lock and cap retainer. The cap retainer acts to prevent the cap from being detached from the neck portion, the force required to pull the cap back over the cap retainer being significantly greater than the force required to move the cap to the cap locked position. In this configuration, the container is sealed, the cap and test plate being retained relative to the container body. The container may then be safely handled by a patient or other user.

To test the sample, the carrier with test plate is removed from the container, the locking features 1019 being operated as needed to release the carrier. The test plate containing the sample may then be analysed. Once the analysis is complete, the container with cap and a swab, and the carrier with test plate may be discarded in a safe manner, either individually, or as a reassembled unit with the carrier returned to engagement with the container body. Alternatively, the container may be cleaned and reused as required.

With reference to FIGS. 16A to 16E, in alternative embodiment, the cap does not have an extension portion. Instead, the cap is provided with an internal recess, extending inside the cap, or through a portion of a thickness of the cap, for receiving and supporting a swab. As described above, the recess may be offset relative to a principal axis of the cap so that rotation of the cap deposits a larger diameter smear on the test plate. It will therefore be appreciated that an offset may be provided either within the cap as in the embodiment of FIGS. 25A & 25B or by providing an offset extension portion as in the embodiment of FIGS. 13 to 24D. Other means for providing a suitable offset to increase a surface area of the smear may be envisaged by the skilled person.

With reference to FIGS. 25A & 25B, in a further alternative embodiment, the cap is provided as a separate component, in this case a C-clip 1021 insertable over the neck portion of the sample container between the cap and a protrusion or poke yoke feature on the body. This may be inserted once the container has been operated to produce a smear on the test plate so as to oppose further movement of the cap along the neck portion and maintain a retained swab out of engagement with the test slide. Other suitable clips and other means for locking the cap relative to the body are envisaged.

The skilled reader will appreciate that embodiments of the present invention may provide a method and apparatus for analysis that can provide swift and accurate analysis of samples, and which will find utility in rapid testing of subjects for certain viruses, among other applications.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

PREFERRED FEATURES OF THE INVENTION

1. A cartridge for use in gathering a swab sample, comprising:
a main body having a sidewall defining an interior space, the main body having an open end;
a swab holder configured to hold a swab, and which is removably attachable to the main body such that at least part of a swab held by the swab holder is within the interior space;
an aperture formed in the sidewall; and
a slide carrier having a slide portion and a rim portion, wherein:
   the slide carrier can be engaged with the aperture in a first position, so that the slide carrier is releasably attached to the sidewall, at least a part of the slide portion is received within the interior space, and at least a part of the rim portion protrudes outwardly beyond the main body; and
   the slide carrier can be repeatedly placed in the first position, and separated from the main body.

2. A cartridge according to clause 1 wherein, when the slide carrier is engaged with the aperture in the main body, all or substantially all of the slide portion of the slide carrier is received within the interior space.

3. A cartridge according to clause 1 or 2 wherein, when the slide carrier is engaged with the aperture in the main body, the slide portion fills or substantially fills the cross-sectional shape of the interior space.

4. A cartridge according to any preceding clause, wherein the rim portion of the slide carrier has a depth which is greater than the depth of the slide portion thereof.

5. A cartridge according to any preceding clause, wherein:
the swab holder is rotatable about a rotational axis with respect to the main body when the swab holder is removably attached to the main body;
the swab holder has a swab holding site at which a swab may be releasably attached to the swab holder; and
the swab holding site is spaced apart from the rotational axis.

6. A cartridge according to any preceding clause, wherein the swab holding site is configured to releasably retain a tubular object.

7. A cartridge according to clause 6, wherein the swab holding site includes one or more gripping walls or protrusions which may releasably retain a tubular object when the tubular object is inserted into the swab holding site.

8. A cartridge according to any preceding clause, further comprising a retaining arrangement to allow the swab holder to be removably attached to the main body.

9. A cartridge according to clause 8, wherein the retaining arrangement comprises cooperating features provided on the cartridge and swab holder.

10. A cartridge according to clause 10, wherein the retaining arrangement comprises cooperating protrusions provided on the cartridge and swab holder.

11. A cartridge according to clause 10, wherein the retaining arrangement comprises cooperating raised ribs provided on the cartridge and swab holder.

12. A cartridge according to any preceding clause, further comprising a spacing member which is selectively attachable to the main body and/or to the swab holder, and wherein when the spacing member is attached to the main body and/or to the swab holder, the relative positions of the main body and holder with respect to one another are limited.

13. A cartridge according to clause 12, wherein the swab holder may, when it is attached to the main body and the spacing member is not attached to the main body, move to within a first distance from the slide carrier, and when the spacing member is attached to the main body, the swab holder may move to within a second distance from the slide carrier, wherein the second distance is greater than the first distance.

14. A cartridge according to any preceding clause, in combination with a swab having a shaft and a head, wherein when the shaft of the swab is held by the swab holder and the swab holder is attached to the main body, the head of the swab may contact the slide portion.

15. A cartridge according to clause 14, when dependent upon clause 15, wherein when the spacing member is attached to the main body, no part of the swab may contact the slide portion.

16. A cartridge according to any preceding claim, when dependent upon one of claims 8-11 wherein, when the swab holder is removably attached to the main body by the retaining arrangement, the swab holder may move through a range of movement with respect to the main body, and wherein at one end of the range of movement the swab holder is relatively close to a slide carrier received in the aperture, and at the other end of the range of movement the swab holder is relatively far away from a slide carrier received in the aperture.

17. A cartridge according to any preceding clause wherein, when the slide carrier is engaged with the aperture, the slide carrier may be removed from the main body by being moved in a first direction, and wherein the slide carrier is wider than the main body in a direction perpendicular with the first direction.

18. A cartridge according to clause 17, wherein the slide carrier comprises engagement features which, when the slide carrier is engaged with the aperture, protrude beyond the main body in a direction perpendicular with the first direction, such that the slide carrier can be removed from the main body by pushing on the engagement features in the first direction.

19. A cartridge according to any preceding claim, wherein the slide portion and rim portion of the slide carrier are formed from different materials.

20. A cartridge according to claim 19, wherein the slide portion is formed from quartz glass.

30. A method of analysing a sample, the method comprising the steps of:
providing a sample on a substrate;
irradiating the sample with two or more incident beams of light, wherein each of the incident beams comprises light of a different frequency, and is transmitted towards the sample along the same or substantially the same axis, and wherein the sample is substantially transmissive at each of the frequencies;
providing a light sensing arrangement to receive light patterns from the incident beams that have passed through the substrate; and
analysing properties of the sample through identifying properties of the light patterns arising from diffraction of the light of the three incident beams by the sample.

31. A method according to clause 30, wherein the sample is irradiated with only two incident beans of light.

32. A method according to clause 30, wherein the sample is irradiated with only three incident beams of light.

33. A method according to clause 30, wherein the sample is irradiated with three or more incident beams of light.

34. A method according to clause 32 or 33, wherein the incident beams comprise light of a first frequency, a second frequency and a third frequency, and wherein the difference in frequency between the first frequency and the second frequency is the same or substantially the same as the difference in frequency between the second frequency and the third frequency.

35. A method according to any one of clauses 30 to 34, wherein each of the incident beams comprises light having a wavelength between 50 nm and 600 nm.

36. A method according to any one of clauses 30 to 35, wherein the sample is irradiated with the incident beams of light sequentially.

37. A method according to any one of clauses 30 to 36, wherein the sample is irradiated with the incident beams of light simultaneously.

38. A method according to any one of clauses 30 to 37, further comprising the steps of:
providing a light pipe with two or more entry guides and an emitting end; and
transmitting the incident beams into respective ones of the entry guides, so that light from each beam is emitted towards the sample from the emitting end along the same or substantially the same axis.

39. A method according to any one of clauses 30 to 38, wherein the step of analysing properties of the sample comprises analysing the light patterns using an image recognition algorithm.

40. A method according to clause 39, wherein the image recognition algorithm comprises a semantic scene segmenting network.

41. A method according to clause 39 or 40, wherein the image recognition algorithm analyses the light patterns to determine whether the sample contains a particular type of object.

42. A method according to any one of clauses 39 to 41, wherein the image recognition algorithm analyses the light patterns to determine whether the sample contains any one of two or more particular types of object.

43. A method according to clause 42, wherein the image recognition algorithm analyses the light patterns two or more times, each time to determine whether the sample contains a particular type of object.

44. A method according to clause 42, when dependent upon clause 40, wherein the network creates a different semantic map for each of the different types of object.

45. A method according to any one of clauses 30 to 44, wherein the step of analysing comprises providing an indication that the sample contains a particular type of object.

46. A method according to clause 45, when dependent upon any of clauses 42 to 44, wherein the step of analysing comprises providing an indication that the sample contains objects of two or more types.

47. A method according to clause 45 or 46, further comprising the step of providing an output that the sample contains a particular virus.

48. A method according to clause 47, comprising the step of distinguishing the particular virus from one or more other types of virus.

49. A method according to any one of clauses 30 to 48, wherein the step of analysing comprises providing an output that the sample does not contain a particular type of object.

50. A method according to clause 49, when dependent upon any of clauses 43 to 44, wherein the step of analysing comprise providing an output that the sample does not contain any of the different types of object.

60. A light pipe, comprising:
a plurality of entry guides, each having a receiving end through which light may be directed into the light pipe;
a junction at which the entry guides converge;
an exit guide extending from the junction and having an emitting end through which light may exit the light pipe.

61. A light pipe according to clause 60 wherein light entering the receiving end of any one of the entry guides will be guided through the junction and the exit guide, and exit the light pipe along the same or substantially the same axis.

62. A light pipe according to clause 60 or 61, wherein the cross-sectional area of the exit guide reduces between the junction and the emitting end.

63. A light pipe according to any one of clauses 60 to 62, comprising first and second entry guides.

64. A light pipe according to clause 63, further comprising a third entry guide.

65. A light pipe according to clause 64, further comprising a fourth entry guide.

66. A light pipe according to any one of clauses 60 to 65, further comprising a reflective coating surrounding at least a part of the entry guides, the junction and the exit guide.

67. A light pipe according to clause 66, wherein the reflective coating covers substantially all of the entry guides, the junction and the exit guide, apart from the receiving ends and the emitting end.

68. A light pipe according to any one of clauses 60 to 67, further comprising a light-absorbing coating surrounding at least a part of the entry guides, the junction and the exit guide.

69. A light pipe according to clause 68, wherein the light-absorbing coating covers substantially all of the entry guides, the junction and the exit guide, apart from the receiving ends and the emitting end.

70. A light pipe according to clause 68 or 69, when dependent upon clause 66 or 67, wherein the light-absorbing coating is formed outside the reflective coating.

80. An analyser, comprising:
a holding arrangement to retain a slide carrier;
an illumination arrangement provided on a first side of the holding arrangement; and
a light sensing arrangement provided on a second side of the holding arrangement, substantially opposite to the first side, wherein:
the illumination arrangement is configured to direct a plurality of incident light beams towards the holding arrangement along the same or substantially the same axis, wherein the incident light beams have different frequencies from each other; and
the light sensing arrangement comprises an array of light sensing elements which are configured to detect light of the frequencies of the incident light beams.

81. An analyser according to clause 80, wherein the holding arrangement has an aperture therethrough, and the axis along which the incident light beams are emitted by the illumination arrangement passes through the aperture.

82. An analyser according to clause 80 or 81, wherein the light sensing arrangement comprises a two-dimensional array of light sensing elements.

83. An analyser according to any one of clauses 80 to 82, wherein the illumination arrangement comprises separate light emitting elements.

84. An analyser according to clause 83, wherein light emitting elements are LEDs.

85. An analyser according to clause 83 to 84, wherein the illumination arrangement comprises first and second light emitting elements.

86. An analyser according to clause 85, wherein the illumination arrangement further comprises a third light emitting element.

87. An analyser according to any one of clauses 83 to 86, wherein the light emitting elements each emit light at different frequencies.

88. An analyser according to clause 86 or 87, wherein the difference between the wavelength of the light emitted by the first light emitting element and the wavelength of the light emitted by the second light emitting element is the same or substantially the same as the difference between the wavelength of the light emitted by the second light emitting element and the wavelength of the wavelength of the light emitted by the third light emitting element.

89. An analyser according to any one of clauses 83 to 88, further comprising a light guide to receive the incident light beams from the light emitting elements and to direct the incident light beams along the axis.

90. An analyser according to clause 89, wherein the light guide comprises a light pipe according to any one of clauses 60 to 70.

91. An analyser according to any one of clauses 80 to 90, further comprising a cartridge holder configured to hold a cartridge, and a removal arrangement configured to remove a slide carrier which is held by a main body of the cartridge and move the slide carrier onto the holding arrangement.

92. An analyser according to clause 91, in combination with a cartridge according to clause 17, wherein the removal arrangement comprises one or more drive elements which are configured to advance and drive the engagement features of the slide carrier to remove the slide carrier from the main body.

93. An analyser according to clause 91 or 92, further comprising a return arrangement, to move the slide carrier from the holding arrangement into engagement with the main body of the cartridge.

94. An analyser according to any one of clauses 80 to 93, further comprising one or more processors on which an image recognition algorithm is implemented, and wherein information from the light sensing arrangement is transmitted to the one or more processors for analysis by the image recognition algorithm.

95. An analyser according to clause 94, wherein the image recognition algorithm is a semantic scene segmenting network.

96. An analyser according to any one of clauses 80 to 94, further comprising a transmission arrangement to transmit information from the light sensing arrangement to a remote location.

100. A method of training an image recognition algorithm, comprising the steps of:
providing an analyser according to any one of clauses 80 to 96;
providing one or more processors arranged to receive information relating to light patterns received by the light sensing arrangement, and being operable to implement an image recognition algorithm;
applying a sequence of samples to respective slide carriers, each of the samples containing one of a known set of viruses;
for each sample, emitting the incident light beams through the slide carrier so that the beams pass through the sample and impinge upon the light sensing arrangement, and transmitting information relating to the light patterns received by the light sensing arrangement to the one or more processors;
training the image recognition algorithm to receive light patterns from an unknown sample containing one of the set of viruses, and to provide a determination as to whether one of the viruses is present, and if so which one of the viruses is present in the sample.

101. A method according to clause 90, wherein the image recognition algorithm is a semantic scene segmenting network.

102. A sample container comprising:
a body defining an internal cavity, the body comprising a neck portion;
a cap engagable with the neck portion so as to close the cavity; and
a test plate removably engagable with the body;
wherein the cap is configured to hold a swab;
wherein the cap is engagable with the neck portion so that a swab is positionable at least partially within the cavity; and
wherein the cap is configured to be slidable along the neck portion so as to adjust a position of a swab within the cavity and relative to the test plate.

103. A sample container according to clause 102, wherein the neck portion comprises a cap retainer for retaining the cap in engagement with the neck portion.

104. A sample container according to clause 102 or 103, wherein the neck portion comprises a cap lock for locking the cap and opposing further sliding movement relative to the neck portion.

105. A sample container according to any of clauses 102 to 104, wherein the cap comprises an annular projection and the cap retainer and cap lock form an annular ring in which the annular projection is receivable so as to opposing further sliding movement of the cap relative to the neck portion.

106. A sample container according to any of clauses 102 to 105, wherein the cap is configured to be rotatable relative to the neck portion so as to rotate a swab within the cavity and relative to the test plate.

107. A sample container according to clause 106, wherein the cap is slidable towards the test plate and rotatable relative to the test plate so as to bring a swab into temporary engagement with the test plate.

108. A sample container according to any of clauses 102 to 107, wherein the cap comprises a recess for receiving a swab.

109. A sample container according to clause 108, wherein the recess is at least partially laterally offset from a centreline of the cap.

110. A sample container according to clause 108 or 109, wherein the recess is formed with a plurality of crush ribs for gripping a swab.

111. A sample container according to any of clauses 102 to 110, wherein a sidewall of the cap is angled to a centreline of the cap.

112. A sample container according to clause 111, wherein the cap comprises a first truncated cone shape.

113. A sample container according to any of clauses 102 to 112, wherein the cap comprises an extension portion.

114. A sample container according to clause 113, where a swab is receivable within the cap extension portion.

115. A sample container according to clause 113 or 114, wherein a sidewall of the cap extension portion is angled to a centreline of the cap extension portion.

116. A sample container according to any of clauses 113 to 115 wherein the cap extension portion comprises a second truncated cone shape.

117. A sample container according to any of clauses 102 to 116, wherein the test plate is insertable into an aperture in the body so as to be at least partially located within the cavity.

118. A sample container according to any of clauses 102 to 117, further comprising a carrier for the test plate, the carrier being is insertable into an aperture in the body so as to support at least a portion of the test plate within the cavity.

119. A sample container according to clause 118, wherein the carrier for the test plate comprises at least one locking feature.

120. A sample container according to any of clauses 102 to 119, wherein the body is generally tubular and closed at an opposite end from the neck portion.

121. A sample container according to any of clauses 102 to 120, wherein the cavity is a substantially cylindrical cavity.

122. A sample container according to any of clauses 102 to 121, wherein the body is integrally formed from plastics material.

123. A sample container according to any of the preceding clauses, wherein the test plate comprises a glass substrate.

124. A cap for sample container comprising:
a cap body defining a recess for receiving a swab;
wherein the recess is provided with at least one crush rib for gripping a swab.

125. A kit of parts comprising:
a sample container as defined in any of clauses 102 to 123; and
a swab.

126. A kit of parts comprising:
a cap for a sample container as defined in clause 124; and
a swab.

1.-40. (canceled)

41. A method of analyzing a sample, the method comprising:
providing a sample on a substrate;
irradiating the sample with at least three incident beams of light, wherein each of the incident beams comprises light of a different frequency, and is transmitted towards the sample along the same or substantially the same axis, and wherein the sample is substantially transmissive at each of the different frequencies;
providing a light sensing arrangement to receive light patterns from the incident beams that have passed through the substrate; and
analyzing properties of the sample through identifying properties of the light patterns arising from diffraction of the light of the three incident beams by the sample,
wherein the incident beams comprise light of a first frequency, a second frequency and a third frequency, and wherein a difference in frequency between the first frequency and the second frequency is the same or substantially the same as a difference in frequency between the second frequency and the third frequency.

42. The method of claim 41, wherein each of the incident beams comprises light having a wavelength between 50 nm and 600 nm.

43. The method of claim 41, wherein the sample is irradiated with the incident beams of light sequentially.

44. The method of claim 41, wherein the sample is irradiated with the incident beams of light simultaneously.

45. The method of claim 41, further comprising:
providing a light pipe with two or more entry guides and an emitting end; and
transmitting the incident beams into respective ones of the entry guides, so that light from each beam is emitted towards the sample from the emitting end along the same or substantially the same axis.

46. The method of claim 41, wherein the step of analyzing properties of the sample comprises analyzing the light patterns using an image recognition algorithm.

47. The method of claim 46, wherein the image recognition algorithm comprises a semantic scene segmenting network.

48. The method of claim 46, wherein the image recognition algorithm analyzes the light patterns to determine whether the sample contains a particular type of object.

49. The method of claim 46, wherein the image recognition algorithm analyzes the light patterns to determine whether the sample contains any one of two or more particular types of object.

50. The method of claim 49, wherein the image recognition algorithm analyses the light patterns two or more times, each time to determine whether the sample contains a particular type of object.

51. The method of claim 50, wherein the image recognition algorithm comprises a semantic scene segmenting network, and wherein the network creates a different semantic map for each different type of object.

52. The method of claim 41, wherein the step of analyzing comprises providing an indication that the sample contains a particular type of object.

53. The method of claim 52, wherein the step of analyzing properties of the sample comprises analyzing the light patterns using an image recognition algorithm, wherein the image recognition algorithm analyzes the light patterns to determine whether the sample contains any one of two or more particular types of object, and wherein the step of analyzing comprises providing an indication that the sample contains objects of two or more types.

54. The method of claim 52, further comprising the step of providing an output that the sample contains a particular virus.

55. The method of claim 54, comprising the step of distinguishing the particular virus from one or more other types of virus.

56. The method of claim 41, wherein the step of analyzing comprises providing an output that the sample does not contain a particular type of object.

57. An analyzer, comprising:
a holding arrangement to retain a slide carrier;
an illumination arrangement provided on a first side of the holding arrangement; and
a light sensing arrangement provided on a second side of the holding arrangement, substantially opposite to the first side, wherein:
the illumination arrangement is configured to direct at least three incident light beams towards the holding arrangement along the same or substantially the same axis, wherein the incident light beams have different frequencies from each other, the illumination arrangement including a first light emitting element, a second light emitting element, and a third light emitting element,
the light sensing arrangement comprises an array of light sensing elements which are configured to detect light of the different frequencies of the incident light beams, and
a difference between a wavelength of the light emitted by the first light emitting element and a wavelength of the light emitted by the second light emitting element is the same or substantially the same as a difference between the wavelength of the light emitted by the second light emitting element and a wavelength of the light emitted by the third light emitting element.

58. The analyzer of claim 57, wherein the holding arrangement has an aperture therethrough, and the axis along which the incident light beams are emitted by the illumination arrangement passes through the aperture.

59. The analyzer of claim 57, wherein the illumination arrangement comprises separate light emitting elements, and the analyzer further comprises a light guide to receive the incident light beams from the light emitting elements and to direct the incident light beams along the axis.

60. The analyzer of claim 57, further comprising one or more processors on which an image recognition algorithm is implemented, and wherein information from the light sensing arrangement is transmitted to the one or more processors for analysis by the image recognition algorithm.
